(19) United States
(12) Patent Application Publication
(10) Pub. No.: US 2011/0173706 A1
(43) Pub. Date: Jul. 14, 2011

(54) NOVEL GPR101 TRANSGENIC MICE AND METHODS OF USE THEREOF

(75) Inventors: Bradford B. Lowell, Southborough, MA (US); Harveen Dhillon, Waltham, MA (US)

(73) Assignee: Beth Israel Deaconess Medical Center, Inc., Boston, MA (US)

(21) Appl. No.: 12/947,157

(22) Filed: Nov. 16, 2010

Related U.S. Application Data
(63) Continuation-in-part of application No. PCT/US2009/003094, filed on May 18, 2009.
(60) Provisional application No. 61/128,110, filed on May 19, 2008.

(51) Int. Cl. A01K 67/027; C12N 5/0735; C12N 15/85; G01N 33/48; G01N 33/567

(52) U.S. Cl. 800/3; 800/18; 435/354; 800/21; 800/24; 800/13; 435/7.21

(57) ABSTRACT

The invention provides transgenic animals comprising a disruption in the endogenous GPR101 gene and methods of producing such transgenic animals. The invention further provides methods of identifying compounds that modulate GPR101 receptor proteins.

### RELATED APPLICATIONS

This application is a continuation-in-part of International Application No. PCT/US2009/003094, which designated the United States and was filed on May 18, 2009, published in English, which claims the benefit of U.S. Provisional Application No. 61/128,110, filed on May 19, 2008. The entire teachings of the above applications are incorporated herein by reference.

### BACKGROUND OF THE INVENTION

G protein receptor 101 (GPR101) is an orphan G protein-coupled receptor (GPCR) with no known ligand. GPR101 is present exclusively in the central nervous system and is abundantly expressed in the hypothalamus and amygdala, specifically in the arcuate nucleus (ARC), ventromedial hypothalamus (VMH), dorsomedial hypothalamus (DMH), posterior hypothalamus (PH), paraventricular nucleus (PVN), medial preoptic area (MPOA), suprachiasmatic (SCN) and anterior hypothalamic area (AHA) of the forebrain regions and the nucleus of the solitary tract (NTS) and lateral parabrachial nucleus (LPB) in the hindbrain regions, areas thought to be involved in metabolic homeostatic function (Nilaweera, K. N. et al., “G Protein-Coupled Receptor 101 mRNA Expression in the Mouse Brain: Altered Expression in the Posterior Hypothalamus and Amygdala By Energetic Challenges,” Journal of Neuroendocrinology, 19: 34-45 (2006), which is incorporated herein by reference in its entirety)). GPR101 is also expressed in areas of the brain involved in regulation of motivated behavior such as the nucleus accumbens in the forebrain and serotonergic nuclei in the midbrain (Bates, et al., “Characterization of GPR101 Expression and G-protein Coupling Selectivity, Brain Research, 1087: 1-14 (2006), which is incorporated herein by reference in its entirety)). Because the hypothalamus, amygdala, LPB and NTS play important roles in energy balance regulation, the high expression of GPR101 mRNA in these regions may suggest important roles for GPR101 in metabolic homeostasis. Therefore, since GPR101 may have an important role in metabolic homeostasis, it may have an important role in treating and preventing metabolic diseases and disorders.

In view of the above, reliable tools to identify the possible physiological consequences of receptor modulation, and to identify potential agents or compositions for use in treatment or prevention of metabolic disease are much needed.

### SUMMARY OF THE INVENTION

The invention features a novel transgenic mouse model, or cells isolated therefrom, for screening agents that modulate the GPR101 receptor. Such models can be used to improve diagnosis of diseases relating to energy metabolism as well as identifying and testing pharmaceutical compositions for better treatment and prevention of disease relating to energy metabolism.

In one embodiment, the invention provides GPR101 knock-out transgenic non-human animals, or cells isolated therefrom, and their use as a model for disease relating to energy metabolism such as obesity, metabolic syndrome, dyslipidemia, insulin resistance syndrome, type 2 diabetes, anorexia nervosa and cachexia. In another embodiment, the invention provides transgenic non-human animals, or cells isolated therefrom, wherein the GPR101 gene is constitutively active and their use as a model for disease relating to energy metabolism such as obesity, metabolic syndrome, dyslipidemia, insulin resistance syndrome, diabetes, particularly type 2 diabetes, anorexia nervosa and cachexia.

In one embodiment the invention relates to a transgenic knock-out non-human mammal whose genome comprises a disruption in the endogenous GPR101 gene.

In another embodiment said disruption has been introduced into the genome by homologous recombination with a DNA targeting construct in an embryonic stem cell.

In one embodiment the disruption of the GPR101 gene results in an inability of said transgenic non-human mammal to produce detectable levels GPR101.

In another embodiment the mammal is a mouse.

The invention further relates to an isolated cell from a transgenic knock-out non-human mammal whose genome comprises a disruption in the endogenous GPR101 gene.

The invention further relates to a method of producing a knock-out non-human mammal whose genome comprises a disruption in the endogenous GPR101 gene.

The invention further relates to a method for screening a candidate agent for the ability to modulate body weight and food intake in a knock-out non-human mammal:
(a) providing a transgenic knock-out non-human mammal whose genome comprises a disruption in the endogenous GPR101 gene;
(b) administering to said knock-out non-human mammal a candidate agent, and
(c) comparing body weight and food intake of transgenic knock-out non-human mammal to the body weight and food intake of a wild-type control; wherein a difference in effect is indicative of an agent that modulates body weight and food intake by altering GPR101 activity.

The invention further relates to a method for screening a candidate agent for the ability to modulate body weight and food intake in a knock-out non-human mammal:
(a) providing a transgenic non-human mammal whose genome comprises a disruption in the endogenous GPR101 gene;
(b) administering to said transgenic non-human mammal a candidate agent; and
(c) evaluating the effect of said candidate agent on the transgenic non-human mammal.

In another embodiment the invention relates to a transgenic non-human mammal whose genome comprises a constitutively active endogenous GPR101 gene.

In one embodiment the constitutively active GPR101 gene has been introduced into the genome by a single amino acid substitution.

In another embodiment the amino acid substitution has been introduced into the genome by homologous recombination with a DNA targeting construct in an embryonic stem cell.

In a further embodiment the mammal is a mouse.

The invention further relates to an isolated cell from a transgenic non-human mammal whose genome comprises a constitutively active endogenous GPR101 gene.

The invention further relates to a method of producing a transgenic non-human mammal whose genome comprises a constitutively active endogenous GPR101 gene.

The invention further relates to a method for screening a candidate agent for the ability to modulate body weight and food intake in a transgenic non-human mammal whose genome comprises a constitutively active endogenous GPR101 gene:
(a) providing a transgenic non-human mammal whose genome comprises a constitutively active endogenous GPR101 gene;
(b) administering to said transgenic non-human mammal a candidate agent, and
(c) evaluating the effect of said candidate agent on the transgenic non-human mammal.

The invention further relates to a method for screening a candidate agent for the ability to modulate the activity of a GPR101 receptor protein, comprising contacting the candidate agent with a GPR101 receptor protein and determining whether the candidate agent binds to the GPR101 receptor protein, wherein a candidate agent that binds to the GPR101 receptor protein is indicative of an agent that modulates the activity of the GPR101 receptor protein.

The invention further relates to a method for screening a candidate agent for the ability to modulate the activity of a GPR101 receptor protein, comprising contacting the candidate agent with a GPR101 receptor protein and determining whether the candidate agent binds to the GPR101 receptor protein, wherein a candidate agent that binds to the GPR101 receptor protein is indicative of an agent that modulates the activity of the GPR101 receptor protein.

### BRIEF DESCRIPTION OF THE DRAWINGS

FIG. 1 shows a three primer multiplex polymerase chain reaction (PCR) strategy to genotype mice.

FIG. 2 is a graph showing body weight in GPR101 knock-out mice (GPR101-KO) and wild-type littermate controls (WT). The Y axis represents body weight in grams (g) and the X axis represents age in weeks (wks).

FIG. 3A is a bar graph of food intake in grams (g) during a twenty-four hour period in GPR101 knock-out mice (GPR101-KO) and wild-type littermate controls (WT).

FIG. 3B is a graph showing cumulative food intake in GPR101 knock-out mice (GPR101-KO) and wild-type littermate controls (WT). The Y axis represents food intake in grams (g) and the X axis represents time in minutes.

FIG. 4 is a graph showing alterations in food consumption for wild-type controls (WT). The Y axis represents food intake in grams (g) and the X axis represents time in minutes.

FIG. 5 is a graph showing alterations in food consumption for GPR101 knock-out mice (GPR101 KO). The Y axis represents food intake in grams (g) and the X axis represents time in minutes.

FIG. 6 is a graph showing body weight of GPR101 knock-out mice (GPR101KO) and wild-type littermate controls (WT) on a chow diet. The Y axis represents body weight in grams (g) and the X axis is age in weeks (wks).

FIG. 7 is a graph showing body weight of GPR101 knock-out mice (GPR101KO) and wild-type littermate controls (WT) on a high fat diet. The Y axis represents body weight in grams (g) and the X axis is age in weeks (wks).

FIG. 8 is a bar graph of body composition (fat mass and lean mass in grams) for mice (wild-type (WT) and GPR101 knock-out mice (GPR101KO) on chow diet.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

### DETAILED DESCRIPTION OF THE INVENTION

The invention relates to transgenic non-human GPR101 knock-out animals, and methods of using the animals for the development of drugs for the treatment or prevention of diseases related to energy metabolism, such as obesity, metabolic syndrome, dyslipidemia, insulin resistance syndrome, diabetes, particularly type 2 diabetes, anorexia nervosa and cachexia. The invention also relates to transgenic non-human animals whose GPR101 gene is constitutively active, and methods of using the animals for the development of drugs for the treatment or prevention of diseases related to energy metabolism, such as obesity, metabolic syndrome, dyslipidemia, insulin resistance syndrome, diabetes, particularly type 2 diabetes, anorexia nervosa and cachexia. Without being bound by a particular mechanism, modulation of the amount or activity of the GPR101 gene may be beneficial in the treatment of such energy metabolism diseases.

### Metabolic Diseases

Metabolic disease include diseases related to energy metabolism, such as, but not limited to, obesity, metabolic syndrome, dyslipidemia, insulin resistance syndrome, diabetes, particularly type 2 diabetes, anorexia nervosa and cachexia.

### Definitions

As used herein, “transgenic non-human animal” includes the founder transgenic non-human animals and progeny of the founders, as well as cells, cell lines and tissues from such animals in which one or more of the cells of the animal includes one or more transgenes. Transgenic non-human animals can be farm animals such as pigs, goats, sheep, cows, horses, and rabbits, rodents such as rats, guinea pigs, and mice, and non-human primates such as baboons, monkeys, and chimpanzees. Transgenic mice are particularly useful. As used herein, a transgene is exogenous DNA which is integrated into the genome of a cell from which a transgenic animal develops and which remains in the genome of the mature animal, thereby directing the expression of an encoded gene product in one or more cell types or tissues of the transgenic animal.

As used herein, a “knock-out” of a gene means an alteration or disruption in the sequence of the gene that results in a decrease of function of the target gene, preferably such that target gene expression is undetectable or insignificant. A knock-out of an endogenous gene means that function of the gene has been substantially decreased so that expression is not detectable or only present at insignificant levels. As used herein the terms “disruption” and “alteration” connote a partial or complete reduction in the expression and/or function of the GPR101 gene. Alteration or disruption of the GPR101 gene can be accomplished by a variety of methods known to those of skill in the art. For example, gene targeting using homologous recombination, mutagenesis (e.g., point mutation) and antisense technology can be used to disrupt a GPR101 gene.

As used herein the term “gene targeting” refers to a type of homologous recombination which occurs as a consequence of the introduction of a targeting construct (e.g., vector) into a mammalian cell (e.g., an ES cell) which is designed to locate and recombine with a corresponding portion of the nucleic acid sequence of the genomic locus targeted for alteration (e.g., disruption) thereby introducing an exogenous recombinant nucleic acid sequence capable of conferring a planned alteration to the endogenous gene. Thus, homologous recombination is a process (e.g., method) by which a particular DNA sequence can by replaced by an exogenous genetically engineered sequence. More specifically, regions of the targeting vector which have been genetically engineered to be homologous (e.g., complimentary) to the endogenous nucleotide sequence of the gene which is targeted for disruption line up or recombine with each other such that the nucleotide sequence of the targeting vector is incorporated into (e.g, integrates with) the corresponding position of the endogenous gene.

As used herein, a “construct” is meant a recombinant nucleic acid, generally recombinant DNA, that has been generated for the purpose of the expression of a specific nucleotide sequence(s), or is to be used in the construction of other recombinant nucleotide sequences.

As used herein, the term “genotype” refers to the genetic makeup of an animal with respect to the GPR101 chromosomal locus. More specifically, the term genotype refers to the status of the animal's GPR101 alleles. GPR101 is located on the X chromosome. Since female mice have two X chromosomes, mice can be of the following three genotypes: wildtype mice (X^{GPR101+}, X^{GPR101+}), heterozygous mice (X^{GPR101+}, X^{GPR101-null}), or homozygous null mice (X^{GPR101-null}, X^{GPR101-null}). In female mice, only one X chromosome is active in any given cell. This X chromosome inactivation is random cell to cell and is often referred to as “lyonization”. Thus, in female mice that are heterozygotes, roughly half the cells in the organism will express no GPR101 (because the X chromosome bearing the wild-type allele (X^{GPR101+}) has been inactivated) while the other half of the cells in the organism will express normal levels of GPR101 (because the X chromosome bearing the “null” allele (X^{GPR101-null}) was inactivated, and hence the X chromosome bearing the wild-type allele (X^{GPR101+}) remains active). Thus, female GPR101 heterozygotes are complex—half of their cells have normal amounts of GPR101 while the other half of their cells will have no GPR101. Wildtype females will always have normal amounts of GPR101 in all cells while homozygous null female mice will always have no GPR101 in all cells. Male mice have one Y chromosome (which lacks a GPR101 gene) and one X chromosome (which has the GPR101 gene). Thus, male mice can only be of one or another genotype: wild-type (Y, X^{GPR101+}) mice which have normal amounts of GPR101 in every cell and knockout (Y, X^{GPR101-null}) mice that lack any GPR101 in every cell.

As a result of the alteration or disruption of the GPR101 gene, the GPR101 knock-out mammal of the present invention can manifest a particular phenotype. As used herein, the term “phenotype” refers to the resulting biochemical or physiological consequences attributed to a particular genotype. In one embodiment, the GPR101 knock-out mammal has altered metabolic homeostasis.

“Knock-out” transgenics can be transgenic animals having a heterozygous knock-out of a gene or a homozygous knock-out of a gene. “Knock-outs” also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

Recombineering (recombination-mediated genetic engineering) is a homologous recombination-based, highly efficient genetic engineering system that can be used to introduce mutations in a target sequence that is part of a vector, such as a BAC. Methods of recombineering are known to those skilled in the art (for example see Zhang et al., Nature Biotech. 18: 1314-7, 2000; Zhang et al., Nature Genetics 20: 123-8 (1998; and Datsenko and Wanner, Proc. Natl. Acad. Sci. USA, 97: 6640-5 (2000). Reviews of recombineering can be found in Court, et al., Annu. Rev. Genet. 36: 361-88 (2002) and Copeland et al., Nature Rev. Genet., 2: 769-779, 2001, which are all incorporated herein by reference in their entirety).

As used herein “constitutively active receptor” shall mean a receptor stabilized in an active state by means other than through binding of the receptor to its ligand or a chemical equivalent thereof A constitutively active receptor may be endogenous or non-endogenous.

“Constitutively activated receptor” shall mean an endogenous receptor that has been modified so as to be constitutively active or to be more constitutively active. “Constitutive receptor activation” shall mean activation of a receptor in the absence of binding to its ligand or a chemical equivalent thereof

The term “ES cell” as used herein refers to pluripotent embryonic stem cells and to such pluripotent cells in the very early stages of embryonic development, including but not limited to cells in the blastocyst stage of development. “Site specific mutagenesis” or “site directed mutagenesis” is a production of a specific predetermined change in a DNA sequence. Methods for site specific mutagenesis can be found in Sambrook et al., Molecular Cloning: A Laboratory Manual, CSH Press 1989, pp. 15.3-15.108; Weiner et al., Gene, 126:35-41 (1993); Sayers et al., Biotechniques, 13: 592-6 (1992); Jones and Winistorfer, Biotechniques, 12: 528-30 (1992); Barton et al., Nucleic Acids Res, 18: 7349-55 (1990); Marotti and Tomich, Gene Anal Tech, 6:67-70 (1989); and Zhu, Anal Biochem, 177: 1204 (1989), which are all incorporated herein by reference in their entirety.

### Methods of Making Transgenic Mice

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191); Brinster et al., Proc. Natl. Acad. Sci. USA 82: 4438-4442 (1985); and in “Manipulating the Mouse Embryo; A Laboratory Manual” 2nd edition (eds., Hogan, Beddington, Costantimi and Long, Cold Spring Harbor Laboratory Press, 1994), which are all incorporated herein by reference in their entirety. Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

Alternatively, the transgenic animals may be obtained by utilizing embryonic stem (ES) cells for the generation of the transgenes. The transgene is introduced into embryonic stem cells and the transfected stem cells are utilized to form an embryo. ES cells are obtained by culturing pre-implantation embryos in vitro under appropriate conditions (Evans et al., Nature 292:154-156 (1981); Bradley et al., Nature 309: 255-256 (1984); Gossler et al., Proc. Acad. Sci. USA 83: 9065-9069 (1986); and Robertson et al., Nature 323: 445-448 (1986), which are incorporated herein by reference in their entirety). The offspring may be analyzed for the integration of the transgene by isolating genomic DNA from tail tissue and the fragment coding for the gene identified by conventional DNA-hybridization techniques (Southern, J. Mol. Biol. 98: 503-517 (1975), which is incorporated herein by reference in its entirety)).

One aspect of the invention pertains to isolating cells or cell lines from the non-human transgenic animals of the invention and growing the cells in culture. Another aspect of the invention pertains to host cells into which a recombinant expression vector of the invention has been introduced, containing sequences which allow it to homologously recombine into a specific site of the host cell's genome, or sequences that allow it to randomly or semi-randomly recombine into the host cell's genome. It is understood that such cells refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Another aspect of the invention pertains to vectors. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated.

Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Such regulatory sequences are described, for example, in Goeddel, Gene Expression Technology: Methods in Enzymology 185, Academic Press, San Diego, Calif. (1990), which is incorporated herein by reference in its entirety. Regulatory sequences include those which direct constitutive expression of a nucleotide sequence in many types of host cells and those which direct expression of the nucleotide sequence only in certain host cells. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of the target gene in prokaryotic or eukaryotic cells. For example, the target gene or fragments can be expressed in bacterial cells such as E. coli, insect cells (using baculovirus expression vectors), yeast cells or mammalian cells. Suitable host cells are discussed further in Goeddel, Gene Expression Technology: Methods in Enzymology 185, Academic Press, San Diego, Calif. (1990), which is incorporated herein by reference in its entirety. Alternatively, the recombinant expression vector can be transcribed and translated in vitro.

The target gene can also be expressed in mammalian cells using a mammalian expression vector. Examples of mammalian expression vectors include pCDM8 (Seed, B., Nature, 329: 840 (1987) and pMT2PC (Kaufman et al., EMBO J., 6: 187 (1987)). When used in mammalian cells, the expression vector's control functions are often provided by viral regulatory elements. For example, commonly used promoters are derived from polyoma, adenovirus 2, cytomegalovirus and Simian virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

A host cell can be any prokaryotic or eukaryotic cell. For example, host cells can be bacterial cells such as E. coli, insect cells, yeast, Xenopus cells, or mammalian cells (such as Chinese hamster ovary cells (CHO), African green monkey kidney cells (COS), or fetal human cells (293T)). Other suitable host cells are known to those skilled in the art. In one aspect of the invention, a host cell is derived from the transgenic non-human animals described herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

### Methods of Using Transgenic Non-Human Animals

The invention provides methods (also referred to herein as “screening assays”) for identifying modulators, i.e., candidate or test compounds or agents (e.g., peptides, cyclic peptides, peptidomimetics, small molecules, small organic molecules, or other drugs) which effect (i.e., modulate, inhibit, reduce, prevent or reverse) diseases related to energy metabolism, such as obesity, metabolic syndrome, dyslipidemia, insulin resistance syndrome, type 2 diabetes, anorexia nervosa and cachexia. As used herein “modulating” or “modulator” or “modulate” refers to agonizing or antagonizing the GPR101 receptor. In particular, transgenic non-human animals of the invention can be used to identify a compound or composition effective for the treatment or prevention of diseases related to energy metabolism. Compounds or compositions can be identified by administering a test compound or composition to a transgenic non-human animal of the invention or by contacting the test compound or composition with an organ, a tissue (e.g., skeletal muscle) or cells (e.g., neuronal cells or muscle cells) derived from the transgenic non-human animal. Effects of the test compound or composition on the energy metabolism on the transgenic non-human animal, organ, tissues or cells are evaluated. For example, a candidate agent can be assessed in the transgenic non-human animals. Test compounds or compositions that alter energy metabolism can be effective for the treatment or prevention of diseases related to energy metabolism.

This invention further pertains to novel agents identified by the above-described screening assays. Accordingly, it is within the scope of this invention to further use test compounds or compositions identified as described herein in an appropriate animal model as described herein. For example, test compounds or compositions identified as described herein can be used in an animal model (e.g., a transgenic GPR101 knock-out non-human animal) to determine the efficacy, toxicity, or side effects of treatment with such test compounds or compositions. Alternatively, test compounds or compositions identified as described herein can be used in an animal model to determine the mechanism of action of such test compounds or compositions.

Test compounds can be formulated into pharmaceutical compositions by admixture with pharmaceutically acceptable non-toxic excipients or carriers and administered to transgenic non-human animals of the invention by any route of administration. For example, parenteral routes such as subcutaneous, intramuscular, intravascular, intradermal, intranasal, inhalation, intrathecal, or intraperitoneal administration, and enteral routes such as sublingual, oral, or rectal administration can be used.

A test compound or composition can be tested for physiologic actions on GPR101 receptors by, for example, administering the test compound or composition to wildtype mice and GPR101 knock-out mice. If the test compound or composition causes an effect in wildtype mice, but not in GPR101 knock-out mice, then the test compound or composition produces the effect by changing the activity GPR101. If the test compound or composition causes loss of body weight, loss of body fat, and/or decreased food intake in wildtype mice, but not in GPR101 knock-out mice, then the test compound or composition is likely to be an activator of GPR101. In contrast, if the test compound or composition causes an increase in body weight, an increase in body fat, and/or an increase in food intake, then test compound or composition is likely to be an inhibitor of GPR101. If the test compound or composition produces an effect in both wildtype mice and GPR101 knock-out mice, then the test compound or composition is not producing its effects by changing GRP101 activity.

The specificity of a test compound or composition can be tested in GPR101 knock-out mice. For example, if effects seen in wildtype mice are also seen in GPR101 knock-out mice, then the test compound or composition is not specific for GPR101.

The GPR101 knock-out mice can be used to determine whether toxicity caused by a test compound or composition is caused by altering GPR101 activity (on-target toxicity) or some other mechanism (off-target toxicity). For example, if a test compound or composition causes a toxic or undesirable effect in wildtype mice but not in GPR101 knock-out mice, then the toxic effect is a byproduct of altering GPR101 activity (an on-target effect). If the test compound or composition causes a toxic or undesirable effect in both wildtype mice and GPR101 knock-out mice, then the toxic effect of the test compound or composition is not related to any changes in the activity of GPR101 (an off-target effect).

The GPR101 constitutively active mice may be particularly useful for testing, for example, a test compound or composition that might decrease the activity of GPR101. Such test compound or composition might be useful for stimulating food intake in patients with anorexia nervosa or cachexia. Since the receptor is activated in these mice, they should be sensitive to potential inhibitors of GPR101 activity.

### In Vitro Screening Methods

In addition to the in vivo and ex vivo screening assays disclosed herein, the invention further provides in vitro methods (also referred to herein as “in vitro screening assays”) for identifying modulators, i.e., candidate or test compounds or agents (e.g., peptides, cyclic peptides, peptidomimetics, small molecules, small organic molecules, or other drugs) which effect (i.e., modulate, inhibit, reduce, prevent or reverse) diseases related to energy metabolism, such as obesity, metabolic syndrome, dyslipidemia, insulin resistance syndrome, type 2 diabetes, anorexia nervosa and cachexia. Such methods can be used to identify compounds or agents that modulate (e.g., agonize, antagonize) the GPR101 receptor.

For example, compounds or agents that modulate the GPR101 receptor can be identified by contacting a test compound or agent with a GPR101 receptor protein in vitro and assessing the ability of the test compound or agent to bind and/or modulate the GPR101 receptor protein. Assays for screening for compounds and agents that bind to and/or modulate the activity of G protein-coupled receptors, including ligand binding assays, activity assays, calcium mobilization assays, cAMP assays, reporter gene assays, ERK phosphorylation assays and β-arrestin assays, are known to those of skill in the art. Exemplary screening assays and high throughput approaches are described, for example, in Xiao, S.-H., et al., Combinatorial Chemistry & High Throughput Screening, 11(9):195-215 (2008), the contents of which are incorporated herein by reference. G protein-coupled receptor assays are also available through commercial sources (e.g., Cisbio US, Bedford, Mass.).

The GPR101 receptor protein employed in such in vitro assays may be, for example, an isolated or recombinant GPR101 receptor protein, or a portion thereof, or may be expressed by an isolated cell or sample of cells (e.g., isolated cells that naturally express a GPR101 receptor, cell lines, recombinant or transgenic host cells), or extracts of such cells.

In a particular embodiment, an agent that binds a GPR101 receptor protein can be identified in a competitive binding assay in which the ability of a test agent to inhibit the binding of GPR101 receptor to a reference agent is assessed. The reference agent can be a full-length GPR101 receptor protein or a portion thereof. The reference agent can be labeled with a suitable label (e.g., radioisotope, epitope label, affinity label (e.g., biotin and avidin or streptavadin), spin label, enzyme, fluorescent group, chemiluminescent group, dye, metal (e.g., gold, silver), magnetic bead) and the amount of labeled reference agent required to saturate the GPR101 receptor protein in the assay can be determined. The specificity of the formation of the complex between the GPR101 receptor protein and the test agent can be determined using a suitable control (e.g., unlabeled agent, label alone).

The capacity of a test agent to inhibit formation of a complex between the reference agent and a GPR101 receptor protein can be determined as the concentration of test agent required for 50% inhibition (IC₅₀ value) of specific binding of labeled reference agent. Specific binding is preferably defined as the total binding (e.g., total label in complex) minus the non-specific binding. Non-specific binding is preferably defined as the amount of label still detected in complexes formed in the presence of excess unlabeled reference agent. Reference agents suitable for use in the method include molecules and compounds which specifically bind to GPR101 receptor, e.g., an antibody that binds GPR101 receptor.

Compounds or agents that bind GPR101 receptors may be evaluated further for GPR101 receptor modulating activity. The ability of a test compound or agent to modulate (e.g., agonize, antagonize) a GPR101 receptor protein can be determined using a suitable receptor activation or inhibition assay. Identified compounds can serve as lead compounds for further diversification using well-known methods of medicinal chemistry. For example, a collection of compounds that are structural variants of the lead can be prepared and screened for GPR101 receptor binding and/or modulating activity. This can result in the development of a structure activity relationship that links the structure of the compounds to biological activity. Compounds that have suitable binding and/or modulatory activity can be developed and tested further for in vivo use.

Candidate modulators can be obtained from one or more available libraries of chemical compounds including, but not limited to, the Chemical Repository of the National Cancer Institute and the Molecular Libraries Small Molecules Repository (PubChem), as well as libraries of the Institute of Chemistry and Cell Biology at Harvard University and other libraries that are available from commercial sources (e.g., Chembridge, Peakdale, CEREP, MayBridge, Bionet). Such libraries or collections of molecules can also be prepared using well-known chemical methods, such as well-known methods of combinatorial chemistry. The libraries can be screened to identify compounds that bind to and/or modulate GPR101 receptor.

This invention further pertains to novel agents identified by the above-described in vitro screening assays. Accordingly, it is within the scope of this invention to further test and use the compounds or compositions identified in the in vitro screening methods described herein in an appropriate animal model as described herein.

A description of example embodiments of the invention follows.

### Exemplification

### Experimental Procedures

### Generation of GPR101 Knock-Out Mice

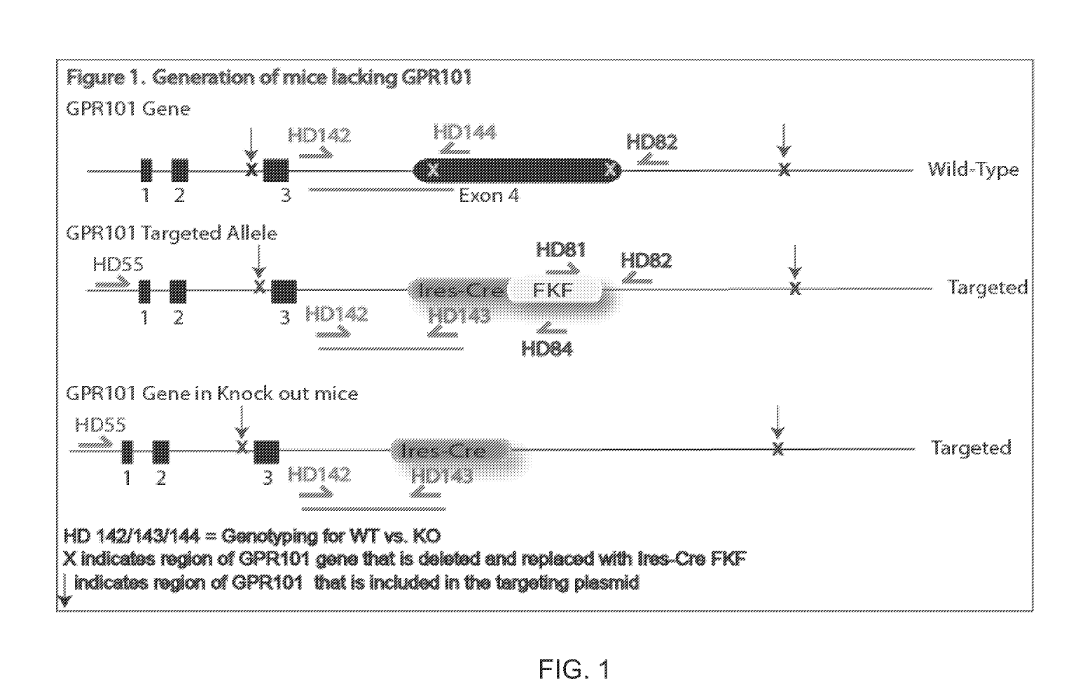

Gene targeting in embryonic stem (ES) cells was used to generate mice lacking GPR101. A replacement targeting construct was prepared using a bacterial artificial chromosome (BAC) genomic clone containing ˜100 kb upstream and ˜40 kb downstream sequence of GPR101. The BAC clone was engineered such that a 2216 by portion of GPR101 sequence (NCBI accession number NC_{—}000086) spanning the coding exon, including the start codon, was removed and replaced with an Ires-Cre-Frt-Kanamycin-Frt (Ires-Cre FKF) cassette. The genomic DNA sequence for GPR101 is in NC_{—}000086 and is from position 54756894 to 54749845 on the assembly. The primers used to engineer the deletion were: (SEQ ID NO: 1) HD73 Forward 5′-TCC TCT GCA AGG CAC TAA CCC TAG CCA CAT GTT TCT CTC GTC CTC AAT CTA GTG ATG TAA TTC CGC CCC TCT CCC T-3′ and (SEQ ID NO: 2) HD74 Reverse 5′-CCT AGC TCC TCA TTT CAG GCT TGC CCT TTT CTG GAT CCC TTT TGA AGA CCT AAA CAA AAT ATT AAC GCT TAC A-3. A 11.4 kb portion of BAC containing the deletion was inserted into pCR-Blunt containing Zeocin. This targeting plasmid was then linearized and electroporated into embryonic stem cells. Targeted clones were identified by PCR analysis using primers spanning the 3′ as well as 5′ end of the targeted locus (FIG. 1). Cells expanded from targeted ES clones were injected into C57BL6 blastocysts and germline transmitting chimeric animals were obtained and then mated with FLPe-recombinase mice. Since GPR101 is located on the X chromosome, the resulting heterozygous offspring were crossed to generate wild-type (+/+) and homozygous (−/−) study subjects. All studies were conducted in male mice. A three primer multiplex PCR strategy was employed to genotype mice as shown in FIG. 1. The following are the primer sequences from FIG. 1:

| | | |
|---|---|---|
| (SEQ ID NO: 3) | HD55 | TTC TTT GCT CCC TCT TCA TTC TCA |
| (SEQ ID NO: 4) | HD81 | CGC ATC GCC TTC TAT CG |
| (SEQ ID NO: 5) | HD82 | ACC TAC TTC ATG TTT ATTT ACG |
| (SEQ ID NO: 6) | HD84 | CGT CAA GAA GGC GAT AGA |
| (SEQ ID NO: 7) | HD142 | TGAGACCCCCAAGAATTAGAAAAA |
| (SEQ ID NO: 8) | HD143 | TTGGCGAGAGGGGAAAGAC |
| (SEQ ID NO: 9) | HD144 | GGGGCCACGAGAGCAACCT |

### Generation of GPR101 Constitutively Active Mice

Mutation of certain residues of GPCRs results in a receptor having an open conformation and hence constitutive activity (activity in the absence of ligand). In order to identify residues within GPR101 that would be involved in potential constitutive activity of the receptor in vitro studies were performed. A full length cDNA encoding mouse GPR101 was amplified from mouse brain RNA and inserted it into an expression plasmid pCDNA 3.1. Site directed mutagenesis was used to identify a mutation in the transmembrane loop of GPR101 that lead to an increase in activity which identify Alanine 397 of GPR101 protein as a target, which when mutated to Lysine, results in a two fold increase in activity of GPR101 (data not shown). A novel mouse model with a single amino acid substitution, A397→K397, was created.

A targeting construct to generate a constitutively activated form of GPR101 with A397→K397 was created using standard techniques involving recombineering. A galK positive and counterselection scheme was used to make the point mutation in the GPR101 BAC (Warming, S. et al., “Simple and Highly Efficient BAC Recombineering Using galK Selection,” Nucleic Acids Research, 33: 1-12 (2005), which is incorporated herein by reference in its entirety). The modified BAC was then inserted into a plasmid and injected into blastocysts for generation of a mouse harboring a point mutation.

### Results

### Body Weight in GPR101 Knock-Out Mice

To determine the functional importance of GPR101 in body weight regulation, GPR101 knock-out mice were generated. GPR101 knock-out mice were fed with standard rodent chow and the body weight weights of male GPR101 knock-out mice (n=8) were compared to those of wild-type littermates (n=12) starting at three weeks of age.

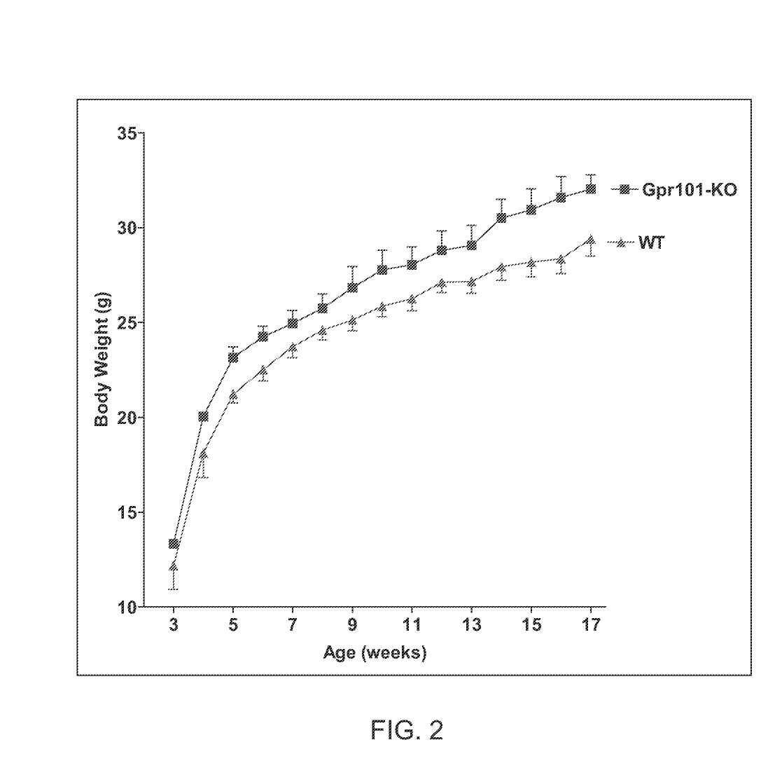

As seen in FIG. 2, male GPR101 knock-out mice trended towards increased body weights compared to wild-type littermate controls. At seventeen weeks of age GPR101 knock-out mice had a ˜10% increase in body weight over that seen in wild-type mice. These results demonstrate that GPR101 in the central nervous system is required for normal body weight homeostasis.

### Food Intake in GPR101 Knock-Out Mice

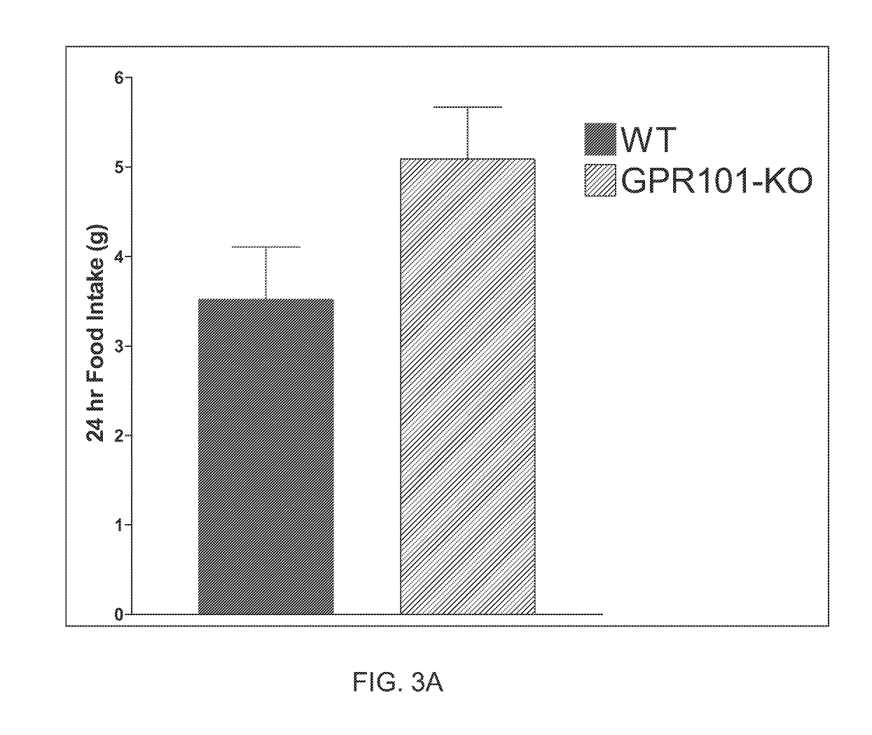

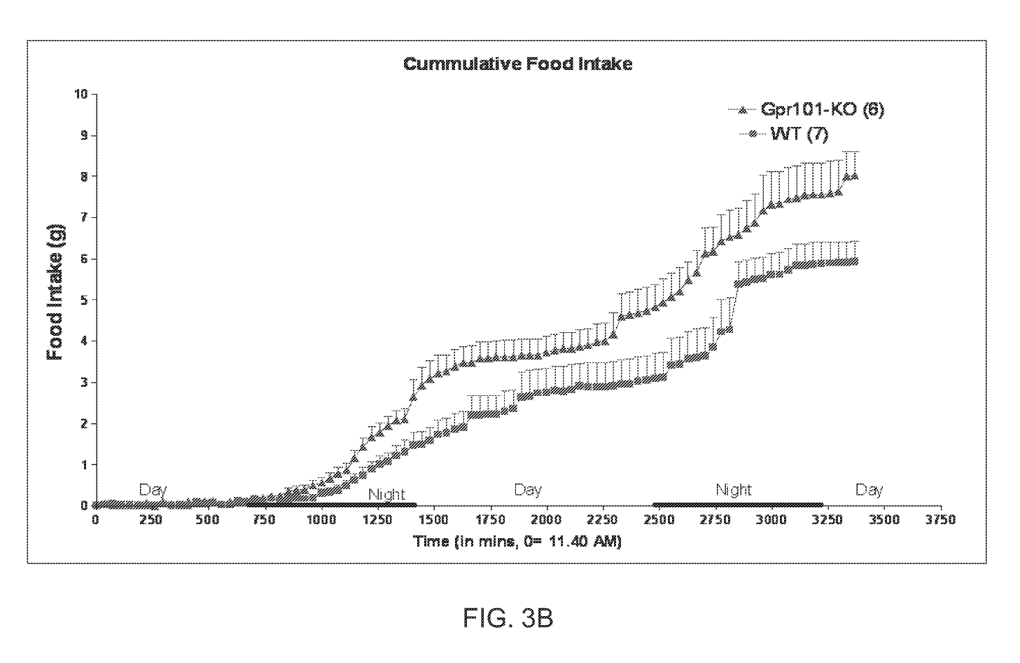

Total food intake was evaluated in GPR101 knock-out mice. GPR101 knock-out mice were fed with standard rodent chow. At six weeks of age, food intake was recorded for a twenty-four hour period (FIG. 3A). The results demonstrate that food intake in a twenty-four hour period was increased in GPR101 knock-out mice as compared with their wild-type littermates (5.1±0.58, GPR101 knock-out vs. 3.52±0.58 wild-type, n=6-7 per group). At eight weeks of age, total food intake was recorded in a comprehensive lab animal monitoring system, and a significant increase in total food consumed by GPR101 knock-out mice was seen as opposed to their wild-type controls (FIG. 3B). The results suggest that GPR101 plays a role in the regulation of meal size.

### Meal Pattern in GPR101 Knock-Out Mice

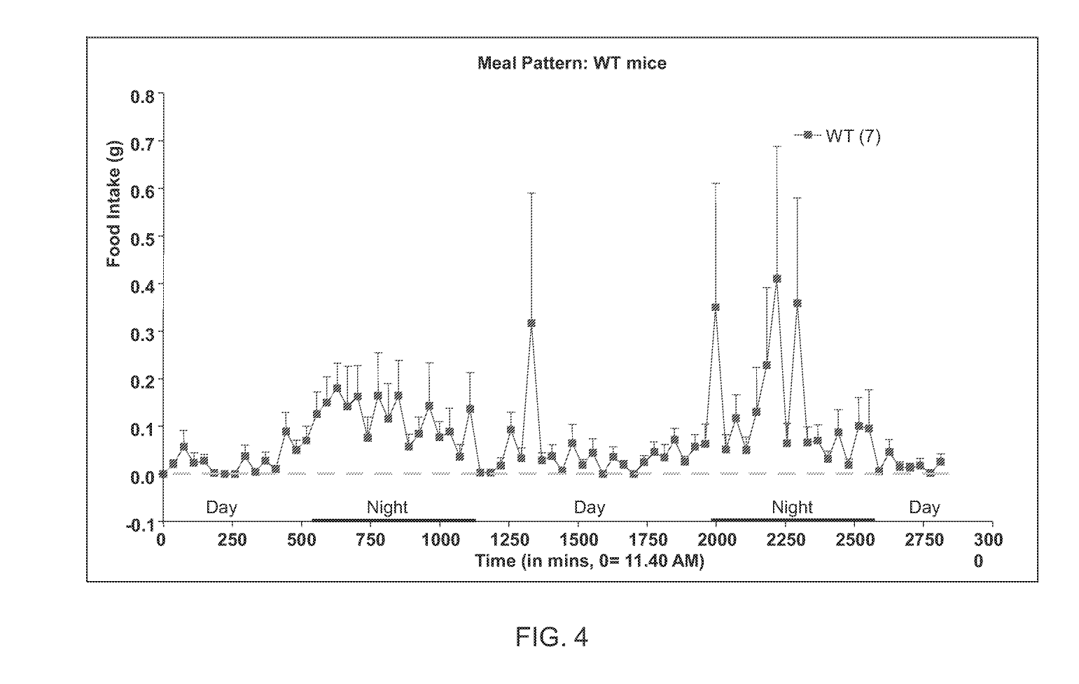

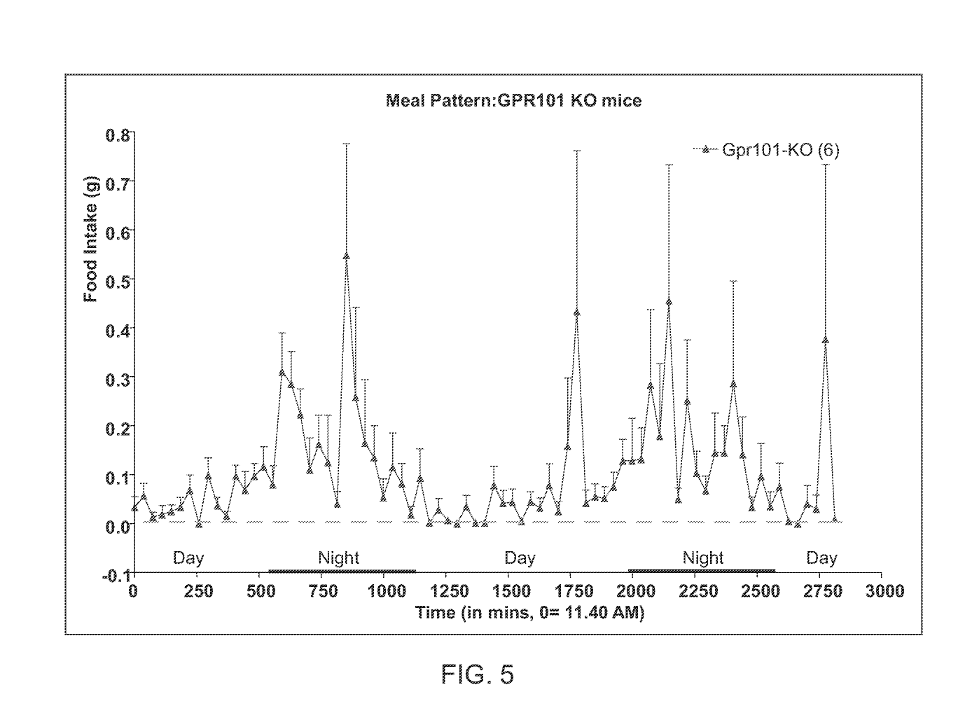

In addition to an increase in twenty-four hour food intake as discussed above, GPR101 knock-out mice at eight weeks of age also exhibit alterations in overall patterns of food consumption as shown in FIG. 5. The GPR101 knock-out mice demonstrated an alteration in meal size as compared to the wild-type controls as shown in FIGS. 4 and 5. The GPR101 knock-out mice consumed larger meals during the dark phase.

The data demonstrates that GPR101 knock-out mice have an increase in body weight and food intake, and, therefore, the GPR101 knock-out mice can be useful in identifying agonists and antagonists of GPR101, which can be used to treat metabolic diseases.

### High Fat Diet Induced Obesity in GPR101 Knock-Out Mice

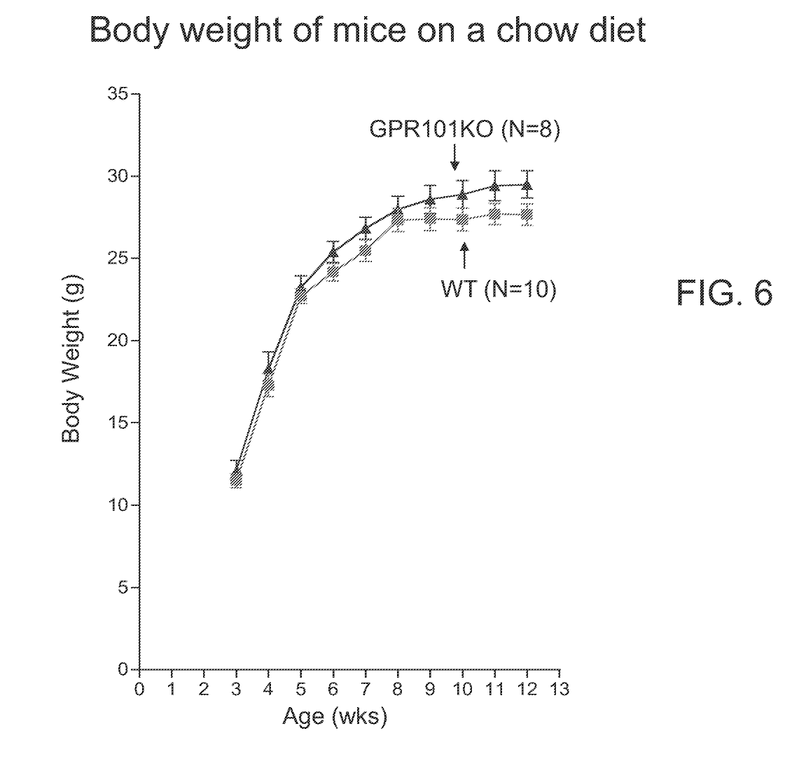

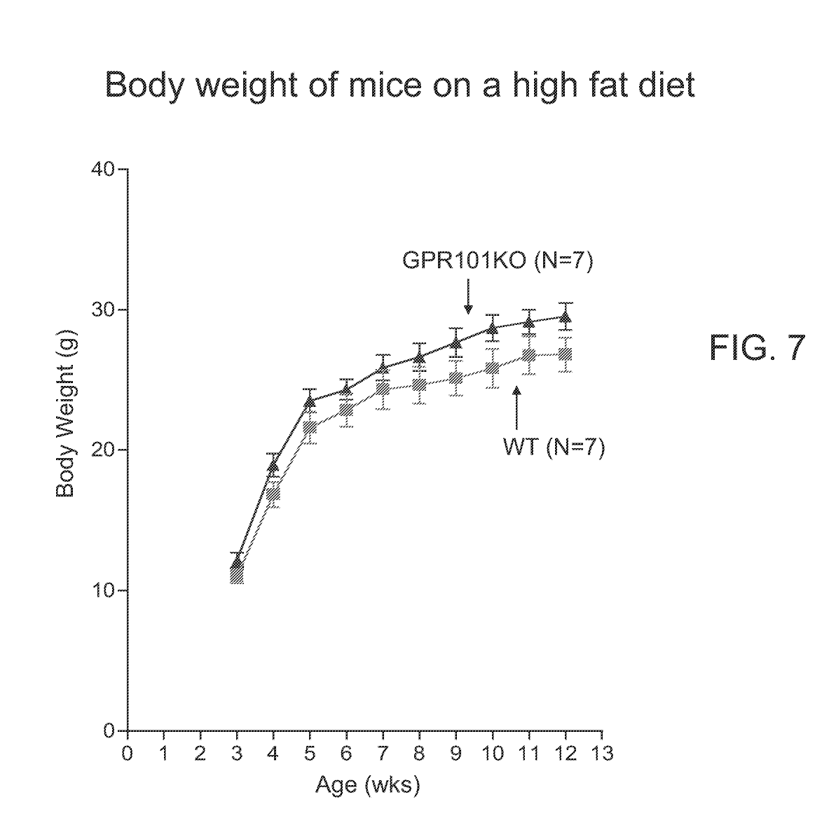

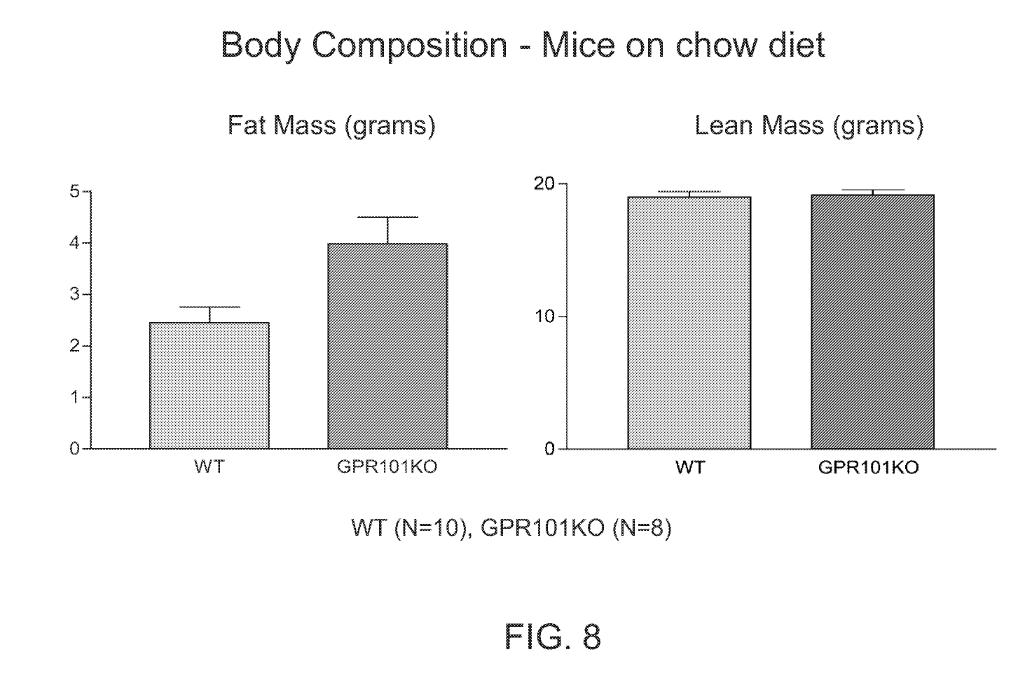

Wildtype and GPR101 gene knock-out mice male mice were placed on a chow diet (Forumulab, diet #5008) (n=10 for WT and n=8 for GPR101-KO) or a high fat diet (45% of calories from fat, Research Diets, D12451) (n=7 for WT and n=7 for GPR101-KO) at weaning (three weeks of age) and body weights were monitored weekly. As previously observed, GPR101KO mice fed a chow diet weigh more than wildtype controls (FIG. 6). Of note, the difference in body weight between wildtype and GPR101KO mice becomes greater when mice are fed a high fat diet (FIG. 7). Body composition of mice fed chow, as assessed by Echo-MRI at twelve weeks of age, indicates that the increase in body weight is due to increased fat mass (FIG. 8). These findings demonstrate that gene knockout of GPR101 induces obesity. In addition, gene knockout of GPR101 causes increased sensitivity to high fat diet-induced obesity. In summary, these findings demonstrate that GPR101 agonists are likely to have anti-obesity effects, and, therefore, could be useful for treating obesity.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A transgenic knock-out non-human mammal whose genome comprises a disruption in the endogenous GPR101 gene.

2. The transgenic non-human mammal of claim 1, wherein said disruption has been introduced into the genome by homologous recombination with a DNA targeting construct in an embryonic stem cell.

3. The transgenic non-human mammal of claim 1, wherein the disruption of the GPR101 gene results in an inability of said transgenic non-human mammal to produce detectable levels GPR101.

4. The transgenic non-human mammal of claim 1, wherein the mammal is a mouse.

5. An isolated cell from a transgenic knock-out non-human mammal whose genome comprises a disruption in the endogenous GPR101 gene.

6. The isolated cell of claim 5, wherein said disruption has been introduced into the genome by homologous recombination with a DNA targeting construct in an embryonic stem cell.

7. The isolated cell of claim 5, wherein the disruption of the GPR101 gene results in an inability of said mouse to produce detectable levels GPR101.

8. The isolated cell of claim 5, wherein the mammal is a mouse.

9. A method of producing a knock-out non-human mammal whose genome comprises a disruption in the endogenous GPR101 gene.

10. The method of claim 9, wherein said disruption has been introduced into the genome by homologous recombination with a DNA targeting construct in an embryonic stem cell.

11. The method of claim 9, wherein the disruption of the GPR101 gene results in an inability of said mouse to produce detectable levels GPR101.

12. The method of claim 9, wherein the mammal is a mouse.

13. A method for screening a candidate agent for the ability to modulate body weight and food intake in a knock-out non-human mammal:
(a) providing a transgenic knock-out non-human mammal whose genome comprises a disruption in the endogenous GPR101 gene;
(b) administering to said knock-out non-human mammal a candidate agent, and
(c) comparing body weight and food intake of transgenic knock-out non-human mammal to the body weight and food intake of a wild-type control; wherein a difference in effect is indicative of an agent that modulates body weight and food intake by altering GPR101 activity.

14. The method of claim 13, wherein said disruption has been introduced into the genome by homologous recombination with a DNA targeting construct in an embryonic stem cell.

15. The method of claim 13, wherein the disruption of the GPR101 gene results in an inability of said mouse to produce detectable levels GPR101.

16. The method of claim 13, wherein the mammal is a mouse.

17. A method for screening a candidate agent for the ability to modulate body weight and food intake in a knock-out non-human mammal:
(a) providing a transgenic non-human mammal whose genome comprises a disruption in the endogenous GPR101 gene;
(b) administering to said transgenic non-human mammal a candidate agent; and
(c) evaluating the effect of said candidate agent on the transgenic non-human mammal.

18. The method of claim 17, wherein said disruption has been introduced into the genome by homologous recombination with a DNA targeting construct in an embryonic stem cell.

19. The method of claim 17, wherein the disruption of the GPR101 gene results in an inability of said mouse to produce detectable levels GPR101.

20. The method of claim 17, wherein the mammal is a mouse.

21. A transgenic non-human mammal whose genome comprises a constitutively active endogenous GPR101 gene.

22. The transgenic non-human mammal of claim 21, wherein the constitutively active GPR101 gene has been introduced into the genome by a single amino acid substitution.

23. The transgenic non-human mammal of claim 22, wherein the amino acid substitution has been introduced into the genome by homologous recombination with a DNA targeting construct in an embryonic stem cell.

24. The transgenic non-human mammal of claim 21, wherein the mammal is a mouse.

25. An isolated cell from a transgenic non-human mammal whose genome comprises a constitutively active endogenous GPR101 gene.

26. The isolated cell of claim 25, wherein the constitutively active GPR101 gene has been introduced into the genome by a single amino acid substitution.

27. The isolated cell of claim 26, wherein the amino acid substitution has been introduced into the genome by homologous recombination with a DNA targeting construct in an embryonic stem cell.

28. The isolated cell of claim 25, wherein the mammal is a mouse.

29. A method of producing a transgenic non-human mammal whose genome comprises a constitutively active endogenous GPR101 gene.

30. The method of claim 29, wherein the constitutively active GPR101 gene has been introduced into the genome by a single amino acid substitution.

31. The method of claim 30, wherein the amino acid substitution has been introduced into the genome by homologous recombination with a DNA targeting construct in an embryonic stem cell.

32. The method of claim 29, wherein the mammal is a mouse.

33. A method for screening a candidate agent for the ability to modulate body weight and food intake in a transgenic non-human mammal whose genome comprises a constitutively active endogenous GPR101 gene:
(a) providing a transgenic non-human mammal whose genome comprises a constitutively active endogenous GPR101 gene;
(b) administering to said transgenic non-human mammal a candidate agent, and
(c) evaluating the effect of said candidate agent on the transgenic non-human mammal.

34. The method of claim 33, wherein the constitutively active GPR101 gene has been introduced into the genome by a single amino acid substitution.

35. The method of claim 34, wherein the amino acid substitution has been introduced into the genome by homologous recombination with a DNA targeting construct in an embryonic stem cell.

36. The method of claim 33, wherein the mammal is a mouse.

37. A method for screening a candidate agent for the ability to modulate the activity of a GPR101 receptor protein, comprising contacting the candidate agent with a GPR101 receptor protein and assessing the activity of the GPR101 receptor protein in the presence of the candidate agent, wherein an alteration in the activity of the GPR101 protein in the presence of the candidate agent is indicative of an agent that modulates the activity of the GPR101 receptor protein.

38. The method of claim 37, wherein the agent agonizes the GPR101 receptor protein.

39. The method of claim 37, wherein the agent antagonizes the GPR101 receptor protein.

40. The method of claim 37, wherein the agent is contacted with an isolated GPR101 receptor protein.

41. The method of claim 37, wherein the agent is contacted with a cell that expresses a GPR101 receptor protein.

42. The method of claim 41, wherein the cell is from a cell line.

43. A method for screening a candidate agent for the ability to modulate the activity of a GPR101 receptor protein, comprising contacting the candidate agent with a GPR101 receptor protein and determining whether the candidate agent binds to the GPR101 receptor protein, wherein a candidate agent that binds to the GPR101 receptor protein is indicative of an agent that modulates the activity of the GPR101 receptor protein.

44. The method of claim 43, wherein the agent is contacted with an isolated GPR101 receptor protein.

45. The method of claim 43, wherein the agent is contacted with a cell that expresses a GPR101 receptor protein.

46. The method of claim 45, wherein the cell is from a cell line.


SEQUENCE LISTING (9 sequences)

SEQ ID NO: 1 - DNA, 76 bp, Artificial Sequence
  Primer
tcctctgcaaggcactaaccctagccacatgtttctctcgtcctcaatctagtgatgtaattccgcccctctccct

SEQ ID NO: 2 - DNA, 73 bp, Artificial Sequence
  Primer
cctagctcctcatttcaggcttgcccttttctggatcccttttgaagacctaaacaaaatattaacgcttaca

SEQ ID NO: 3 - DNA, 24 bp, Artificial Sequence
  Primer
ttctttgctccctcttcattctca

SEQ ID NO: 4 - DNA, 17 bp, Artificial Sequence
  Primer
cgcatcgccttctatcg

SEQ ID NO: 5 - DNA, 22 bp, Artificial Sequence
  Primer
acctacttcatgtttatttacg

SEQ ID NO: 6 - DNA, 18 bp, Artificial Sequence
  Primer
cgtcaagaaggcgataga

SEQ ID NO: 7 - DNA, 24 bp, Artificial Sequence
  Primer
tgagacccccaagaattagaaaaa

SEQ ID NO: 8 - DNA, 19 bp, Artificial Sequence
  Primer
ttggcgagaggggaaagac

SEQ ID NO: 9 - DNA, 19 bp, Artificial Sequence
  Primer
ggggccacgagagcaacct